(19) United States
(12) Patent Application Publication
(10) Pub. No.: US 2023/0255792 A1
(43) Pub. Date: Aug. 17, 2023

(54) SURGICAL TOOL FOR EXPANDABLE CAGE

(71) Applicant: ENDAC CO.,LTD., Gyeonggi-do (KR)

(72) Inventors: Kyung Hak KIM, Gyeonggi-do (KR); Gil Sam LEE, Gyeonggi-do (KR)

(21) Appl. No.: 18/003,010

(22) PCT Filed: Jul. 7, 2021
(86) PCT No.: PCT/KR2021/008625

§ 371 Date: Dec. 22, 2022

(30) Foreign Application Priority Data
Jul. 8, 2020 (KR) 10-2020-0084049

(51) Int. Cl. A61F 2/46; A61F 2/44

(52) U.S. Cl. CPC A61F 2/4611; A61F 2/4455; A61F 2002/4627; A61F 2002/4687; A61F 2002/30579

(57) ABSTRACT

A surgical tool for an expandable cage, the surgical tool including a hollow guide part, a first end of which is coupled to the cage, and a second end of which has a display part on which a numerical expansion value of the cage is displayed, and a driving part which is inserted into the guide part, the driving part being configured to rotate by being coupled to a shaft of the cage at an end thereof so as to expand the cage. According to the surgical tool for an expandable cage, the guide part is provided with the display part such that the numerical expansion value of the cage can be checked from the outside, thereby enabling stable surgery.

### CROSS REFERENCE TO RELATED APPLICATIONS

This is a continuation of International Application No. PCT/KR2021/008625 filed on Jul. 7, 2021, which claims priority from Korean Patent Application No. 10-2020-0084049 filed on Jul. 8, 2020. The contents of these applications are incorporated herein by reference in their entireties.

### TECHNICAL FIELD

The present disclosure relates generally to a surgical tool for an expandable cage. More particularly, the present disclosure relates to a surgical tool for an expandable cage which can vertically expand the cage inserted into space between neighboring vertebrae to adhere the vertebrae to each other and to appropriately adjust a distance therebetween.

### BACKGROUND

Generally, a disc present between vertebrae functions as a joint and plays a very important role in minimizing impact applied to the vertebrae as the position and shape of nucleus pulposus accommodated inside the disc changes according to the movement of the vertebrae. Most of the nucleus pulposus is composed of water, but as the amount of water gradually decreases with age, the disc loses a buffering function thereof.

Accordingly, when excessive pressure is applied to fibers, back pain occurs, and when the pressure further progresses, the fibers are severely stretched or ruptured, pressing on a nerve root located at a back side and causing pain in the pelvis and legs. Moreover, various side effects may occur, such as the gradual decreasing of a distance between the vertebrae or the deformity of the vertebrae as the vertebrae collapse.

As a method of treating disease caused by a damaged disc, there is a method of replacing space between two adjacent vertebrae with a prosthetic material, i.e., a so-called cage, after removing the damaged intervertebral disc. That is, the cage functions to restore a distance between two adjacent vertebral bodies, which is the original height of an intervertebral disc, so as to restore a spinal function.

The cage for vertebrae can be inserted into space between the vertebrae to provide a bone formation inducing material (bone powder) for adhering the vertebrae to each other. As a special type of cage, an expandable cage is provided so that parts thereof can be moved in a vertical direction so that the height of the cage can be adjusted appropriately according to a distance between the vertebrae. In this case, it is preferable to have a means by which the bone formation inducing material (bone powder) filled in the cage can be replenished. A technology concerning such a cage proposed by an applicant of the present disclosure is disclosed in Korean Patent No. 10-2106024.

The cage for vertebrae can be expanded by using a surgical tool for a cage. That is, the surgical tool for a cage is coupled to one end of the expandable cage and expands the cage inserted into space between vertebrae, and a bone formation inducing material (bone powder) is injected into the expanded cage to inject the bone formation inducing material (bone powder) in the space between vertebrae.

However, in a case in which the applicant of the present disclosure expands the expandable cage by using the conventional surgical tool for a cage, the cage is configured to expand while being inserted into space between vertebrae, and thus it is difficult to accurately determine the degree of expansion of the cage. In addition, a device for accurately guiding the surgical tool for a cage, which is moved and coupled to the vertebrae from the outside, is complicated, and thus the use efficiency of the surgical tool is reduced.

### BRIEF SUMMARY

The present disclosure has been made to solve the above problems occurring in the prior art and is intended to propose a surgical tool for an expandable cage in which a numerical expansion value of the cage can be checked from the outside to enable a stable surgery, and the cage can be expanded by the simple structure of the surgical tool such that the use efficiency of the surgical tool is increased.

In order to accomplish the above objectives, according to an embodiment of the present disclosure, a surgical tool for an expandable cage includes: a hollow guide part, a first end of which is coupled to the cage, and a second end of which has a display part on which a numerical expansion value of the cage is displayed; and a driving part which is inserted into the guide part, the driving part being configured to rotate by being coupled to a shaft of the cage at an end thereof so as to expand the cage.

Here, the guide part may include a cylindrical guide body having threads formed on an outer circumferential surface of a first end of the guide body such that the threads of the guide body are coupled to threads formed on an inner circumferential surface of a block recess of the cage.

In addition, the display part may be provided on a second end of the guide body and may display the numerical expansion value of the cage by corresponding to the rotation of the driving part.

Furthermore, the display part may include: a display body which is formed to have a cylindrical shape and is provided on the second end of the guide body, the display body having a plurality of display holes formed along an outer circumferential surface thereof; a cylindrical rotation bar which is inserted into the display body and rotates together with the driving part, the rotation bar having threads formed on an outer circumferential surface thereof in a longitudinal direction thereof; and a display means which is screwed to the rotation bar and is moved along the longitudinal direction of the rotation bar by the rotation of the driving part.

In this case, the rotation bar may have a plurality of coupling grooves formed along an inner circumferential surface of an end part thereof, and the driving part may have a plurality of coupling protrusions formed on an outer circumferential surface thereof by protruding therefrom such that the coupling protrusions have shapes corresponding to the coupling grooves so as to be coupled to the coupling grooves.

In addition, an inner diameter of the guide part and an outer diameter of the driving part may correspond to each other such that the driving part moves in a straight line along a longitudinal direction of the guide part.

Meanwhile, the surgical tool for an expandable cage of the present disclosure may further include: a hollow fixing part into which the guide part is inserted, the fixing part being configured to grip the cage at an end thereof. Here, the fixing part may include: a cylindrical fixing part body; and grip parts which are formed on a first end of the fixing part body by protruding therefrom and are inserted into grip recesses of the cage so as to grip the cage.

In addition, the fixing part may further include a fixing handle formed on an outer circumferential surface of a second side of the fixing part body by protruding therefrom.

Furthermore, the surgical tool for an expandable cage of the present disclosure may further include: a bone powder guide member including a cylindrical bone powder guide which is selectively inserted into the guide part, and a press part which is selectively inserted into the bone powder guide and presses bone powder injected into the bone powder guide.

According to the surgical tool for an expandable cage of the present disclosure, the guide part is provided with the display part such that the numerical expansion value of the cage, which is disposed between vertebrae and is expanding, can be checked from the outside, thereby enabling stable surgery, and a plurality of numerical value graduations of the display part is provided along the outer circumferential surface of the display body such that the numerical expansion value can be checked from multiple directions during surgery, thereby making the surgical tool convenient.

In addition, the inner diameter of the guide part and the outer diameter of the driving part correspond to each other, and thus the driving part can move in a straight line along the longitudinal direction of the guide part and can be rapidly and accurately coupled to the cage due to a simple structure of the driving part, thereby having high efficiency of use.

Furthermore, after the cage is expanded by using the driving part, the bone powder guide member can be inserted into the guide part so as to directly inject a bone formation inducing material (bone powder) to the cage, thereby increasing the use efficiency of the surgical tool.

### DESCRIPTION OF DRAWINGS

FIG. 1 is a perspective view illustrating a cage coupled to a surgical tool for an expandable cage of the present disclosure,

FIG. 2 is a perspective view of the surgical tool for an expandable cage according to one embodiment of the present disclosure,

FIG. 3 is a perspective view of a fixing part of the surgical tool for an expandable cage illustrated in FIG. 2,

FIG. 4 is a perspective view of a guide part of the surgical tool for an expandable cage illustrated in FIG. 2,

FIG. 5 is an exploded perspective view illustrating a display part of the guide part illustrated in FIG. 4,

FIG. 6 is a perspective view of the display part illustrated in FIG. 5,

FIG. 7 is a sectional view of the display part illustrated in FIG. 6,

FIG. 8 is a perspective view of a driving part of the surgical tool for an expandable cage illustrated in FIG. 2, and

FIG. 9 is a perspective view of a bone powder guide member inserted into the guide part of the surgical tool for an expandable cage illustrated in FIG. 2.

### DETAILED DESCRIPTION

Hereinafter, an exemplary embodiment according to the present disclosure will be described in detail with reference to the accompanying drawings. Prior to this, terms or words used in this specification and claims should not be construed as being limited to usual or dictionary meaning, and based on the principle that an inventor can adequately define the concept of the terms in order to best describe his or her invention, the terms must be interpreted as meanings and concepts consistent with the technical idea of the present disclosure.

Accordingly, the embodiment described in this specification and the configurations shown in the drawings are for only the most exemplary embodiment of the present disclosure, and do not represent all of the technical ideas of the present disclosure. Therefore, it should be understood that at the time of this application, there may be equivalent modified embodiments that can replace the embodiment.

Hereinafter, an embodiment of a surgical tool for an expandable cage according to the present disclosure will be described in detail with reference to the accompanying drawings.

The surgical tool 10 for an expandable cage according to the present disclosure is a surgical tool for vertically expanding the expandable cage C inserted into space between neighboring vertebrae.

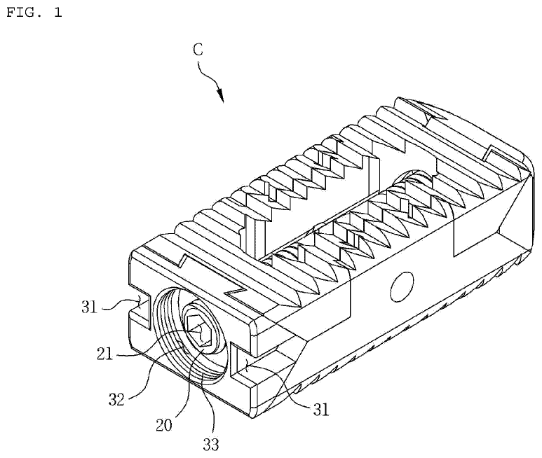

In this case, as illustrated in FIG. 1, the expandable cage C may be vertically expanded by moving a cage block connected to a shaft 20 due to the rotation of the shaft 20. A grip recess 31 may be formed in each of the opposite sides of the cage block, a block recess 32 may be formed in the cage block, and the threads 33 of the block recess may be formed along the inner circumferential surface of the block recess 32. In addition, a shaft hole 21 is formed in an end of the shaft 20 and thus the surgical tool 10 for an expandable cage may be connected to the shaft 20 through the shaft hole 21. That is, when the surgical tool 10 for an expandable cage connected to the shaft 20 rotates, the shaft 20 is rotatable. In this case, in FIG. 1, the cross section of the shaft hole 21 is illustrated to have a hexagonal shape but is not limited thereto, and may be appropriately changed to correspond to the surgical tool 10 for an expandable cage. The shaft 20 may have a flow path formed therein so as to inject the bone formation inducing material (bone powder) thereto. The flow path may communicate with the shaft hole 21 to receive and deliver the bone formation inducing material (bone powder) to the shaft and the inside of the cage C. Meanwhile, the expandable cage C is illustrated as an expandable cage which expands in a vertical direction, but may be applied to an expandable cage which expands in vertical and horizontal directions, and a detailed description of the expandable cage is omitted.

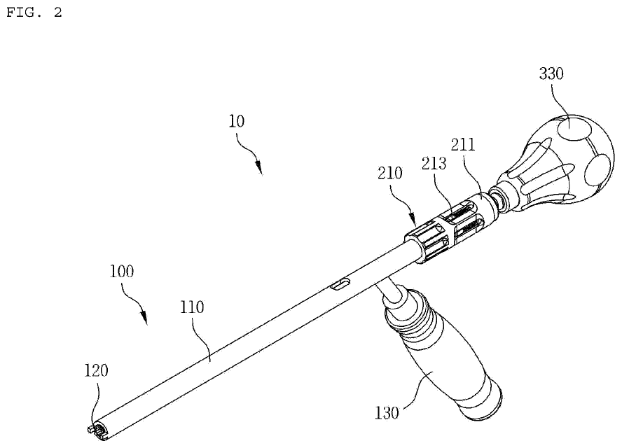

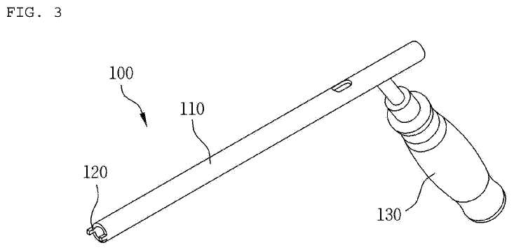

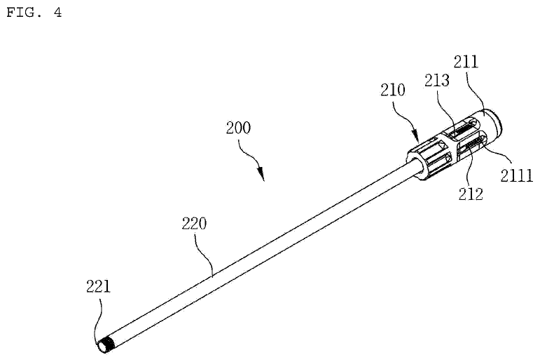

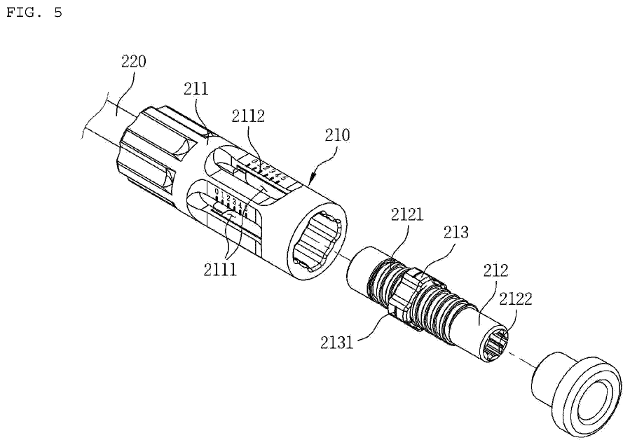

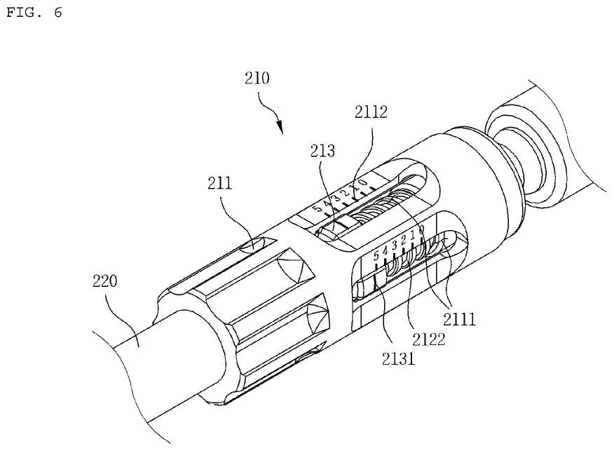

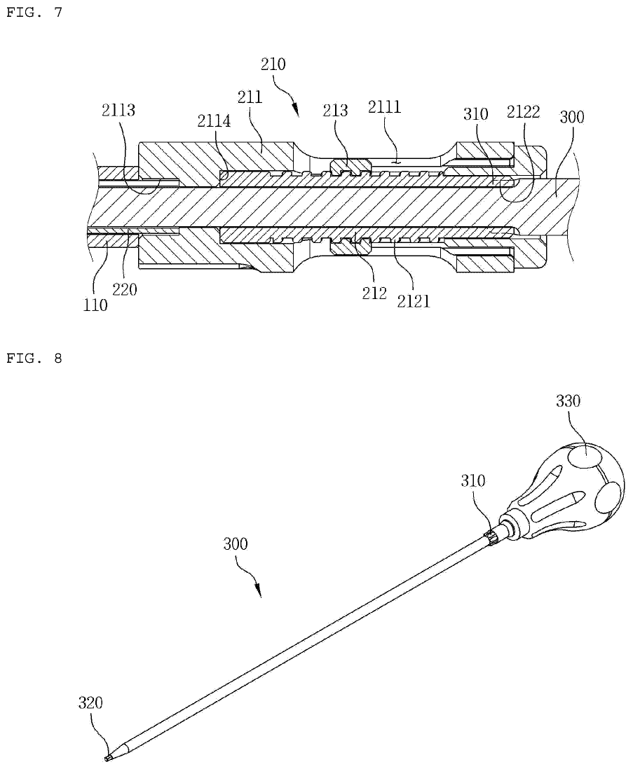

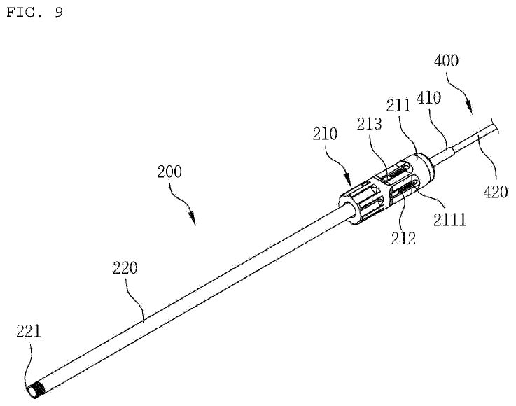

Referring to FIGS. 2 to 9, the surgical tool 10 for an expandable cage includes a guide part 200, a driving part 300, a fixing part 100, and a bone powder guide member 400.

The guide part 200 is intended to guide the driving part 300 to be described later, and a first end of the guide part 200 may be coupled to the cage C. The guide part 200 includes a guide body 220 and a display part 210.

The guide body 220 is formed to be hollow. The guide body 220 has threads 221 formed on an outer surface of a first end thereof and is screwed to the threads 33 formed on the inner circumferential surface of a block recess 32 of the cage C. That is, the first end of the guide body 220 is formed to correspond to the block recess 32 and thus may be screwed to the threads 33 of the block recess 32. The threads 221 of the first end of the guide body 220 and the threads 33 of the block recess 32 are screwed to each other, so the guide body 220 can be stably fixed to the cage C.

The display part 210 is provided on the second end of the guide body 220. The display part 210 displays a numerical expansion value of the cage C, and the numerical expansion value of the cage C expanding by corresponding to the rotation of the driving part 300 can be checked through the display part. The display part 210 may include a display body 211, a rotation bar 212, and a display means 213.

The display body 211 is formed to have a cylindrical shape and is provided on the second end of the guide body 220. The display body 211 has a plurality of display holes 2111 formed along an outer circumferential surface thereof. The plurality of display holes 2111 is preferably formed along the longitudinal direction of the display body 211. The display body 211 may be fixedly connected to the second end of the guide body 220. In other words, a recess 2113 into which the second end of the guide body 220 can be inserted is preferably formed in the first end of the display body 211. Accordingly, the guide body 220 is inserted into and fixedly coupled to the recess 2113.

The display body 211 has numerical value graduations 2112 formed along the longitudinal direction thereof such that the numerical expansion value of the cage C expanding can be checked. The numerical value graduations 2112 are displayed along the longitudinal direction of each of the display holes 2111 on the surface of the display body 211, and are preferably formed in the plurality of display holes 2111. In other words, the numerical value graduations 2112 are preferably displayed such that the numerical expansion value increases gradually toward the second side of the display body 211 from the first side thereof.

The rotation bar 212 is inserted into the display body 211. The rotation bar 212 is rotatably provided and rotates together with the driving part 300. The rotation bar 212 has threads 2121 formed on an outer circumferential surface thereof in a longitudinal direction thereof and is formed to have a cylindrical shape. More specifically, the rotation bar 212 is provided to be rotatable by being disposed on a step 2114 formed on the inner circumferential surface of the guide body 220, and may rotate together with the driving part 300 due to the rotation of the driving part 300. Meanwhile, the rotation bar 212 preferably has a plurality of coupling grooves 2122 formed on an inner circumferential surface thereof.

The display means 213 is coupled to the threads of the rotation bar 212 and moves along the longitudinal direction of the rotation bar 212 due to the rotation of the driving part 300. The display means 213 is formed into a ring shape, and is screwed to the threads 2121 of the rotation bar 212. That is, the display means 213 has screw grooves formed on an inner circumferential surface thereof such that the screw grooves correspond to the threads 2121 of the rotation bar 212 and thus the display means 213 and the rotation bar 212 can be screwed to each other. The display means 213 moves according to the rotation of the rotation bar 212, and the numerical expansion value of the cage C can be checked through the moving distance of the display means 213. In other words, an indicator line 2131 is marked on the outer surface of the display means 213, and through a position at which the indicator line 2131 moved by the movement of the display means 213 corresponds to a numerical value graduation 2112, the numerical expansion value of the cage C can be checked. The display means 213 is provided in space between the display body 211 and the rotation bar 212 and may move along the threads 2121 of the rotation bar 212 by being pressed in contact with the inner surface of the display body 211. More specifically, the display means 213 is formed to protrude in a portion of the outer circumferential surface thereof at each position corresponding to the display hole 2111, and thus the portion of the display means 213 is preferably disposed to protrude from the display hole 2111. Accordingly, a portion of the display means 213 is disposed by being held in the display hole 2111, and does not rotate together with the rotation bar 212 and can move in the longitudinal direction of the rotation bar 212 along the threads of the rotation bar 212.

The driving part 300, which is a component to expand the cage C, is inserted into the guide part 200, and a first end of the driving part 300 is coupled to the shaft 20 of the cage C. The driving part 300 rotates while the first end thereof is coupled to the shaft 20 and thus can expand the cage C. That is, when the driving part 300 is rotated, the shaft 20 connected thereto is rotated, and due to the structure of the cage C, the space of the cage C may expand, and according to the amount of the rotation of the driving part 300, the numerical expansion value of the cage C may be adjusted. The driving part 300 is configured to have an outer diameter corresponding to the inner diameter of the guide part 200. Accordingly, the driving part 300 may move in a straight line along the longitudinal direction of the guide part 200 and may be easily coupled to the shaft 20. In other words, the driving part 300 may be coupled to the shaft 20 by moving to the first end of the guide part 200 from a second end thereof. The driving part 300 may move in a straight line along the longitudinal direction of the guide part 200 and may be rapidly coupled to the shaft 20 located at the first side of the guide part 200 due to the simple structure of the driving part 300. The driving part 300 may include a plurality of coupling protrusions 310, a rotation coupling body 320, and a turning handle 330.

The plurality of coupling protrusions 310 is configured to be coupled to the coupling grooves 2122, and is formed on the outer circumferential surface of the driving part 300 by protruding therefrom such that the coupling protrusions 310 have shapes corresponding to the coupling grooves 2122. The plurality of coupling protrusions 310 may be formed by being spaced apart from each other at predetermined intervals along the circumference of the driving part 300 and may be coupled to the plurality of coupling grooves 2122, respectively. Accordingly, since the plurality of coupling protrusions 310 and the plurality of coupling grooves 2122 are coupled to each other, the driving part 300 and the rotation bar 212 can rotate together.

The rotation coupling body 320 is intended to be coupled to the shaft hole 21, and is inserted into and engaged with the shaft hole 21 to be coupled thereto. That is, the rotation coupling body 320 may be formed into a shape corresponding to the shaft hole 21 to be coupled thereto. The shaft hole 21 is preferably formed to have a hexagonal cross section, and the rotation coupling body 320 is also preferably formed to have a hexagonal cross section by corresponding to the cross section of the shaft hole 21. However, the cross sections of the shaft hole 21 and the rotation coupling body 320 are not limited to the hexagonal shapes, but may be appropriately changed into other shapes in which the shaft hole 21 and the rotation coupling body 320 are engaged with each other by corresponding to each other.

The turning handle 330 may be provided on the second end of the driving part 300. The turning handle 330 is intended to rotate the driving part 300, and a user may expand the cage C by grasping the turning handle 330 and rotating the driving part 300.

The fixing part 100 is formed to be hollow, has guide part 200 inserted thereinto, and grips the cage C at a first end thereof. To this end, the fixing part 100 includes a fixing part body 110, grip parts 120, and a fixing handle 130.

The fixing part body 110 is formed to have a cylindrical shape and is fitted over the guide part 200.

The grip parts 120 are intended to grip the cage C and are formed on a first end of the fixing part body 110 by protruding therefrom. That is, the grip parts 120 are preferably formed on a portion of the first end of the fixing part body 110 by protruding therefrom, and the protruding grip parts 120 are inserted respectively into the grip recesses 31 of the cage C. The grip parts 120 are formed by protruding to correspond to the shape of the grip recesses 31 of the cage C and are inserted into the grip recesses 31 so as to grip the opposite ends of the cage C. In other words, before the guide part 200 is screwed to the block recess 32 of the cage C, the grip parts 120 are preferably coupled to the grip recesses 31. That is, after the fixing part 100 and the cage C are coupled to each other through the grip parts 120 and the grip recesses 31, the guide part 200 is preferably inserted into the fixing part body 110 such that the guide part 200 and the cage C are screwed to each other. The grip parts 120 may respectively grip the opposite ends of the cage C such that impact occurring during the insertion of the cage C into space between vertebrae can be distributed without concentrating on the cage C, and may stably support and easily move the cage C when adjusting the position of the cage C by moving the cage C vertically and horizontally inside the vertebrae.

The fixing handle 130 is provided on the second side of the fixing part body 110 and is formed on the outer circumferential surface of the fixing part body 110 by protruding therefrom. When expanding the cage C by using the surgical tool 10 for an expandable cage, a user may grip the fixing handle 130 and may securely fix the position of the surgical tool 10 for an expandable cage.

The bone powder guide member 400 is intended to fill the bone formation inducing material (bone powder) in the flow path formed in the shaft 20 and includes a bone powder guide 410 and a press part 420.

The bone powder guide 410 is formed into a cylindrical shape and is selectively inserted into the guide part 200. The bone powder guide 410 is hollow to guide the bone formation inducing material (bone powder) to be filled, and is inserted into the guide part 200. After the driving part 300 is separated from the guide part 200 after expanding the cage C through the driving part 300, the bone powder guide 410 is inserted into the guide part 200.

The press part 420 may be selectively inserted into the bone powder guide 410 and may press the bone formation inducing material (bone powder) injected into the bone powder guide 410. That is, after the bone formation inducing material (bone powder) is injected into the bone powder guide 410 after inserting the bone powder guide 410 into the guide part 200, the press part 420 may press the bone formation inducing material (bone powder) to be moved to the shaft 20. Accordingly, the press part 420 may press the bone formation inducing material (bone powder) injected inside the bone powder guide 410 by moving along the longitudinal direction thereof and may inject the bone formation inducing material into the space of the cage C expanded by the driving part 300. That is, after expanding the cage C by using the driving part 300, the bone powder guide member 400 may be inserted into the guide part 200 to directly inject a bone formation inducing material (bone powder) into the cage C, thereby increasing the use efficiency of the surgical tool.

Hereinafter, the process of using the surgical tool 10 for an expandable cage will be described step by step.

First, the grip parts 120 are respectively inserted into the grip recesses 31 of the cage C such that the fixing part 100 is coupled to the cage C.

Next, the guide part 200 is coupled to the cage C. In this case, the position of the indicator line 2131 of the display means 213 is required to be located at a zero point of the graduations of the display part 210 of the guide part 200. That is, after coupling the guide part 200 and the driving part 300 to each other, the driving part 300 is rotated such that the position of the indicator line 2131 of the display means 213 is adjusted to be disposed at the zero point (0) of the numerical value graduations 2112. In addition, while the guide body 220 is inserted into the fixing part body 110, the guide body 220 is coupled to the cage C. The guide body 220 is inserted into the fixing part body 110 through the second side of the fixing part body 110, and is moved from the second side of the fixing part body 110 to the first side thereof such that the guide body 220 can be screwed to the block recess 32 of the cage C. In other words, while the guide part 200 is inserted into the fixing part 100, the threads 221 formed on the guide body 220 and the threads 33 formed on the inner circumferential surface of the block recess 32 are screwed to each other, so the guide part 200 may be stably fixed to the cage C.

Next, by inserting the driving part 300 into the guide part 200, the rotation coupling body 320 and the shaft hole 21 of the shaft 20 are coupled to each other to be engaged with each other, and simultaneously, the coupling protrusions 310 is coupled to the coupling grooves 2122 to be inserted thereto. In this case, the outer diameter of the driving part 300 and the inner diameter of the guide part 200 correspond to each other, and thus the driving part 300 can move in a straight line along the longitudinal direction of the guide part 200, so the rotation coupling body 320 can be easily coupled to the shaft 20.

Next, the cage C is expanded by rotating the driving part 300. In this case, the display means 213 moves along the longitudinal direction of the rotation bar 212 by correspond to the amount of the rotation of the driving part 300, so the numerical expansion value of the cage C according to the position of the indicator line 2131 can be checked from the outside. The plurality of numerical value graduations 2112 is provided along the outer circumferential surface of the display body 211 and thus the numerical expansion value of the cage C can be checked from multiple directions, so the numerical expansion value of the cage C inserted into space between vertebrae is easily checked from the outside.

When the cage C is expanded by rotating the driving part 300 such that the numerical expansion value of the cage C reaches a preset appropriate numerical expansion value, the driving part 300 is separated from the guide part 200, and then the bone powder guide member 400 is inserted into the guide part 200 such that the bone formation inducing material (bone powder) is filled in the flow path formed in the shaft 20. That is, after inserting the bone powder guide 410 into the guide part 200, the bone formation inducing material (bone powder) is injected into the bone powder guide 410, and then the press part 420 may press the bone formation inducing material (bone powder) to move to the shaft 20. Accordingly, after expanding the cage C by using the driving part 300, the bone powder guide member 400 is inserted into the guide part 200 to directly the bone formation inducing material (bone powder) to the cage, thereby increasing the use efficiency of the surgical tool.

In the surgical tool 10 for an expandable cage according to the present disclosure, the guide part 200 is provided with the display part 210 such that the numerical expansion value of the cage C, which is disposed between vertebrae and is expanding, can be checked from the outside, thereby enabling stable surgery, and the plurality of numerical value graduations 2112 of the display part 210 is provided along the outer circumferential surface of the display body 211, so the numerical expansion value can be checked from multiple directions during surgery, thereby making the surgical tool convenient.

In addition, the inner diameter of the guide part 200 and the outer diameter of the driving part 300 correspond to each other, and thus the driving part 300 can move in a straight line along the longitudinal direction of the guide part 200 and can be rapidly and accurately coupled to the cage C due to the simple structure of the driving part, thereby having high efficiency of use.

Furthermore, after the cage C is expanded by using the driving part 300, the bone powder guide member 400 can be inserted into the guide part 200 so as to directly inject a bone formation inducing material (bone powder) to the cage, thereby increasing the use efficiency of the surgical tool.

The present disclosure has been described with reference to the embodiment shown in the drawings, but this is only exemplary, and it should be understood that those skilled in the art may make various modifications and equivalent other embodiments. Accordingly, the technical scope of protection of the present disclosure should be determined by the technical spirit of the appended claims.

<Description of the Reference Numerals in the Drawings>

| | | | |
|---|---|---|---|
| 10: | Surgical tool for an expandable cage | 110: | Fixing part body |
| 100: | Fixing part | 130: | Fixing handle |
| 120: | Grip part | 210: | Display part |
| 200: | Guide part | 2111: | Display hole |
| 211: | Display body | 2113: | Recess for insertion |
| 2112: | Numerical value graduations | 212: | Rotation bar |
| 2114: | Step | 2122: | Coupling groove |
| 2121, 221: | Thread | 2131: | Indicator line |
| 213: | Display means | 300: | Driving part |
| 220: | Guide body | 320: | Rotation coupling body |
| 310: | Coupling protrusion | 400: | Bone powder guide member |
| 330: | the turning handle | 420: | Press part |
| 410: | Bone powder guide | 20: | Shaft |
| C: | Cage | 31: | Grip recess |
| 21: | Shaft hole | 33: | Threads of block recess |
| 32: | Block recess | | |

1. A surgical tool for an expandable cage, the surgical tool comprising:
a hollow guide part, a first end of which is coupled to the cage, and a second end of which has a display part on which a numerical expansion value of the cage is displayed; and
a driving part which is inserted into the guide part, the driving part being configured to rotate by being coupled to a shaft of the cage at an end thereof so as to expand the cage.

2. The surgical tool of claim 1, wherein the guide part comprises a cylindrical guide body having threads formed on an outer circumferential surface of a first end of the guide body such that the threads of the guide body are coupled to threads formed on an inner circumferential surface of a block recess of the cage.

3. The surgical tool of claim 2, wherein the display part is provided on a second end of the guide body and displays the numerical expansion value of the cage by corresponding to the rotation of the driving part.

4. The surgical tool of claim 3, wherein the display part comprises:
a display body which is formed to have a cylindrical shape and is provided on the second end of the guide body, the display body having a plurality of display holes formed along an outer circumferential surface thereof;
a cylindrical rotation bar which is inserted into the display body and rotates together with the driving part, the rotation bar having threads formed on an outer circumferential surface thereof in a longitudinal direction thereof; and
a display means which is screwed to the rotation bar and is moved along the longitudinal direction of the rotation bar by the rotation of the driving part.

5. The surgical tool of claim 4, wherein the rotation bar has a plurality of coupling grooves formed along an inner circumferential surface of an end part thereof, and
the driving part has a plurality of coupling protrusions formed on an outer circumferential surface thereof by protruding therefrom such that the coupling protrusions have shapes corresponding to the coupling grooves so as to be coupled to the coupling grooves.

6. The surgical tool of claim 1, wherein an inner diameter of the guide part and an outer diameter of the driving part correspond to each other such that the driving part moves in a straight line along a longitudinal direction of the guide part.

7. The surgical tool of claim 1, further comprising:
a hollow fixing part into which the guide part is inserted, the fixing part being configured to grip the cage at an end thereof.

8. The surgical tool of claim 7, wherein the fixing part comprises:
a cylindrical fixing part body; and
grip parts which are formed on a first end of the fixing part body by protruding therefrom and are inserted into grip recesses of the cage so as to grip the cage.

9. The surgical tool of claim 8, wherein the fixing part further comprises a fixing handle formed on an outer circumferential surface of a second side of the fixing part body by protruding therefrom.

10. The surgical tool of claim 1, further comprising:
a bone powder guide member comprising a cylindrical bone powder guide which is selectively inserted into the guide part, and a press part which is selectively inserted into the bone powder guide and presses bone powder injected into the bone powder guide.
